(19) United States
(12) Patent Application Publication
(10) Pub. No.: US 2025/0316380 A1
(43) Pub. Date: Oct. 9, 2025

(54) SYSTEMS AND METHODS FOR DERIVATION OF PATIENT-SPECIFIC TUMOR DYNAMICS USING A SPECTRAL ANALYSIS OF AN IMAGE

(71) Applicant: H. LEE MOFFITT CANCER CENTER AND RESEARCH INSTITUTE, INC., Tampa, FL (US)

(72) Inventors: Heiko ENDERLING, Tampa, FL (US); Stefano PASETTO, Tampa, FL (US)

(21) Appl. No.: 18/866,264

(22) PCT Filed: May 16, 2023
(86) PCT No.: PCT/US2023/022328

§ 371 Date: Nov. 15, 2024

Related U.S. Application Data
(60) Provisional application No. 63/342,298, filed on May 16, 2022.

(51) Int. Cl. G16H 50/20; A61N 5/10; G06T 7/00

(52) U.S. Cl. CPC G16H 50/20; A61N 5/1048; G06T 7/0012; A61N 2005/1074; G06T 2207/30024; G06T 2207/30096

(57) ABSTRACT

An example computer-implemented method for determining patient-specific tumor dynamics is described herein. The method includes receiving an image of a tissue sample that includes a distribution of tumor cells and non-tumor cells; applying a spatial correlation function to the image; and obtaining a power spectral density of the spatial correlation function of the image. The method also includes fitting the power spectral density of the spatial correlation function of the image to the power spectrum of a reaction-diffusion model; and inferring a patient-specific tumor-dynamic coefficient based on the reaction-diffusion model of cancer.

### CROSS-REFERENCE TO RELATED APPLICATIONS

This application claims the benefit of U.S. provisional patent application No. 63/342,298, filed on May 16, 2022, and titled “SYSTEMS AND METHODS FOR DERIVATION OF PATIENT-SPECIFIC TUMOR DYNAMICS USING A SPECTRAL ANALYSIS OF AN IMAGE,” the disclosure of which is expressly incorporated herein by reference in its entirety.

### BACKGROUND

Cancer is a polymorphic system that evolves over many scales, from intracellular through signaling pathways, macromolecular trafficking, intercellular through cell-cell adhesion and communication, and tissue level through cell-extracellular matrix interaction mechanical forces (1). The complex interaction and co-evolution of the tumor with the host immune system recently received significant attention from cancer therapies that boost the immune system's antitumor response. Therefore, it is imperative to characterize tumor topology and heterogeneity and its overall growth and invasion potential in the complex tissue environment.

Great attention has been given to a mathematical formalization of growth and diffusion processes for cancer (2). The most significant mathematical advancement in using continuous diffusion models to study cancer dynamics comes from brain cancer modeling (3-6). General studies of tissue invasion (hypotaxis) analyze wave solutions and the tracking of heat-shock proteins (7), or hypoxic cell waves and the pseudo palisades (hypercellular regions) in glioblastoma (GBM) (8) in connection with imaging (9). Similarly, the morphing of the extracellular matrix architecture by breast cancer cell diffusion and infiltration has been demonstrated (10). Diffusion equations have mainly been used in compartment model approaches, e.g., for evolution from low to high-grade glioma (11) and its response to radiotherapy fractionation (12). More recently, reaction-diffusion equations have been deployed on the cellular level to simulate spatial cell size distributions and their influence on uptake rates (13-15).

In most of these works, diffusion models are assumed as an instrument to interpret a clinical condition (e.g., the anamnesis of a patient, a longitudinal past collection of data, etc.) and forecast tumor dynamics with and without therapeutic interventions. A remarkable example is a study of DTI-MRI derived brain biomechanics that can predict the location of secondary cancer foci distant to the primary tumor (16).

No less important is the role of the diffusion process models in the approach to the clinically significant problems of patient-specific imaging-derived predictive models in response to neoadjuvant therapy of breast cancer. This problem was studied by several authors (17-22), focusing on the coupling diffusion model to extracellular matrix stiffness, in connection with radiotherapy (23-25) or synergy of radiation therapy and chemotherapy (26), surgical resection (27), necrosis density thresholds (28), radiation-induced necrosis (29), in vitro treatment of triple-negative breast cancer cell lines (30), in connection with in vitro growth (31) or synthetic models of solid tumors (32).

### SUMMARY

Systems and methods for deriving patient-specific tumor dynamics are described herein. For example, systems and methods for deriving cancer patient-specific reaction-diffusion rates from spectral analysis of immunohistochemistry slides are described herein.

An example computer-implemented method for determining patient-specific tumor dynamics is described herein. The method includes receiving an image of a tissue sample that includes a distribution of tumor cells and non-tumor cells; applying a spatial correlation function to the image; and obtaining a power spectral density of the spatial correlation function of the image. The method also includes fitting the power spectral density of the spatial correlation function of the image to a power spectrum of a reaction-diffusion model; and inferring a patient-specific tumor-dynamic coefficient based on the reaction-diffusion model of cancer.

In some aspects, the patient-specific tumor-dynamic coefficient is a growth rate or a diffusion rate.

In some aspects, the spatial correlation function is a 2-point correlation function or a 2-point cross-correlation function.

In some aspects, the step of obtaining the power spectral density of the spatial correlation function of the image includes applying a Fourier transform to the spatial correlation function of the image.

In some aspects, the image is an immunofluorescence stained slide image, an immunohistochemistry (IHC) stained slide image, or a hematoxylin & eosin (H&E) stained slide image.

In some aspects, the method further includes simulating a tumor's response to treatment using the patient-specific tumor-dynamic coefficient.

In some aspects, the cancer is breast cancer, head and neck cancer, or prostate cancer.

In some aspects, the tissue sample is a pre-treatment sample or a post-treatment sample.

In some aspects, the step of receiving the image of the tissue sample includes receiving a single image, and the patient-specific tumor-dynamic coefficient is derived from the reaction-diffusion model of cancer using the single image.

An example method of treating cancer in a subject is described herein. The method includes determining patient-specific tumor dynamics for the subject according to the techniques described herein; and guiding a treatment regimen for the subject using the patient-specific tumor-dynamic coefficient.

In some aspects, the step of guiding the treatment regimen for the subject includes increasing or decreasing an amount or frequency of treatment administered to the subject.

In some aspects, the treatment regimen is one or more of chemotherapy, immunotherapy, or radiotherapy.

In some aspects, the step of guiding the treatment regimen for the subject includes selecting one or more of chemotherapy, immunotherapy, or radiotherapy based on the one or more patient-specific tumor-dynamic coefficients.

In some aspects, the treatment regimen is a combination therapy.

In some aspects, the method further includes administering the treatment regimen to the subject.

An example system for determining patient-specific tumor dynamics is also described herein. The system includes: at least one processor; and at least one memory operably coupled to the at least one processor, wherein the at least one memory has computer-executable instructions stored thereon that, when executed by the at least one processor, cause the at least one processor to: receive an image of a tissue sample including a distribution of tumor cells and non-tumor cells; apply a spatial correlation function to the image; obtain a power spectral density of the spatial correlation function of the image; fit the power spectral density of the spatial correlation function of the image to a power spectrum of a reaction-diffusion model; and infer a patient-specific tumor-dynamic coefficient based on the reaction-diffusion model of cancer.

In some aspects, the patient-specific tumor-dynamic coefficient is a growth rate or a diffusion rate.

In some aspects, the spatial correlation function is a 2-point correlation function or a 2-point cross-correlation function.

In some aspects, the step of obtaining the power spectral density of the spatial correlation function of the image includes applying a Fourier transform to the spatial correlation function of the image.

In some aspects, the image is an immunofluorescence stained slide image, an immunohistochemistry (IHC) stained slide image, or a hematoxylin & eosin (H&E) stained slide image.

In some aspects, the at least one memory has further computer-executable instructions stored thereon that, when executed by the at least one processor, cause the at least one processor to simulate a tumor's response to treatment using the patient-specific tumor-dynamic coefficient.

In some aspects, the cancer is breast cancer, head and neck cancer, or prostate cancer.

In some aspects, the tissue sample is a pre-treatment sample or a post-treatment sample.

In some aspects, the step of receiving the image of the tissue sample includes receiving a single image, and the patient-specific tumor-dynamic coefficient is derived from the reaction-diffusion model of cancer using the single image.

Another example computer-implemented method for determining patient-specific tumor dynamics is described herein. The method includes receiving a single image of a tissue sample that includes tumor cells; analyzing the single image; and deriving patient-specific tumor-dynamic information based on the analysis.

Yet another computer-implemented method for determining patient-specific tumor dynamics is described herein. The method includes receiving a medical image of tissue that includes a distribution of tumor cells and non-tumor cells; applying a spatial correlation function to the medical image; and obtaining a power spectral density of the spatial correlation function of the medical image. The method also includes fitting the power spectral density of the spatial correlation function of the medical image to a power spectrum of a reaction-diffusion model; and inferring a patient-specific tumor-dynamic coefficient based on the reaction-diffusion model of cancer.

In some aspects, the medical image is a magnetic resonance image (MRI), positron emission tomography (PET) image, or a computer tomography (CT) image.

An example computer-implemented method for classifying tumor cells is also described herein. The method includes receiving an image of a tissue sample that includes a distribution of tumor cells and non-tumor cells; applying a spatial correlation function to the image; and classifying one or more cells in the image as a tumor or non-tumor cell using the spatial correlation function.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A is a pictorial representation of tissue resection areas process. FIG. 1B illustrates stained images (left column), cell localization (central column), and 2pCF (right column) for the tumoral, interface, and stromal ROI (top, central and bottom rows, respectively). The brown dashed curve in the top panel of the right column shows the 2pCF for only cancer cells. Red is used for the tumor and green for all the other components in the localization plot column. In the third column, the characteristic trends of the 2pCF for ROI with the presence of cancer (solid, positive correlation), for the interface areas (dropping curve with anticorrelation in the outer ROI), and for the stroma ROI (small positive correlation with random irregular areas of anticorrelation) bordering the noise frequency detection thresholds are seen.

FIG. 2A illustrates (left column) spatial distribution monochromatic-stained for some patient-#20 tumoral resection areas (sites A, B, and C, the first three rows) referring to pre-treatment biopsy. On the central column, the corresponding 2pCF, on the right column the PSD. FIG. 2B illustrates (left column) spatial distribution monochromatic-stained for some patient-#20 tumoral resection areas (sites D, E, and F, the first three rows) referring to post-treatment biopsy. On the central column, the corresponding 2pCF, on the right column the PSD.

FIG. 3 illustrates PSD pre- and post-treatment for several patients. The solid curve refers to the reaction-diffusion (RD) model fit to the three pre-SBRT ROI (A, B, C; red) and the three post-SPRT ROI (D, E, F; blue). Dashed curves are the averages, and transparent shaded areas show standard error.

FIGS. 4A-4C illustrate a comparison of pre- and post-SABR reaction-diffusion parameters. FIG. 4AA illustrates spatially averaged across all [symbol] ROI per-patient diffusion parameters, [symbol] ₚₐₜ, normalized by the time-averaged patient-specific diffusion parameter, [symbol] , across all [symbol] ROI for all times (pre and post). FIG. 4B is similar to FIG. 4A but for the growth rates.

FIG. 4C is similar to FIG. 4A but for ratio of the two coefficients. FIGS. 4D-4E presents the first probability distribution function for diffusion coefficients (FIG. 4D) and reaction rates (FIG. 4E) for the whole set of 53 patients.

FIG. 5 is a flowchart illustrating example operations for determining patient-specific tumor dynamics according to implementations described herein.

FIG. 6 is an example computing device.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” an, “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for stained histopathological slides sampled from breast cancer patients, it will become evident to those skilled in the art that the implementations are not limited thereto, but are applicable for other slide images and/or other cancers.

As used herein, the terms “about” or “approximately” when referring to a measurable value such as an amount, a percentage, and the like, is meant to encompass variations of ±20%, ±10%, ±5%, or ±1% from the measurable value.

“Administration” of “administering” to a subject includes any route of introducing or delivering to a subject an agent. Administration can be carried out by any suitable means for delivering the agent. Administration includes self-administration and the administration by another.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The term “tumor” is defined herein as an abnormal mass of hyperproliferative or neoplastic cells from a tissue other than blood, bone marrow, or the lymphatic system, which may be benign or cancerous. In general, the tumors described herein are cancerous. As used herein, the terms “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of solid cancerous growths, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. Examples of solid tumors are sarcomas, carcinomas, and lymphomas. Leukemias (cancers of the blood) generally do not form solid tumors.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Examples include, but are not limited to, lung carcinoma, adrenal carcinoma, rectal carcinoma, colon carcinoma, esophageal carcinoma, prostate carcinoma, pancreatic carcinoma, head and neck carcinoma, or melanoma. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Cancer is a prevalent disease, and while many significant advances have been made, the ability to accurately predict how an individual tumor will grow—and ultimately respond to therapy—remains limited. Tumor growth and invasion destroy the natural biological architecture of cells organized in healthy tissues. Traditionally, signatures of this morphological transformation have been investigated and quantified using spatial statistics analyses. We develop a theoretical framework to connect spatial tissue analyses with tumor growth and invasion. We exploit spatial-spectral analyses to connect the morphological changes induced by the cancer growth, as observed in histopathological tissue slides, to the parameters in the reaction-diffusion equation expressing the growth and invasion. We compare power-spectral density from the data-deduced two-point correlation function to the corresponding theoretical power distribution predicted by the reaction-diffusion equation. We find that a histopathological slide, taken at a single time-point—such as routinely collected from biopsies at cancer diagnosis—suffices to interpret disease dynamics into the framework of the reaction-diffusion equation. This novel approach may tackle both model-parameter-inference problems for tumor-infiltration forecasting and tumor classification.

Understanding tumor growth and invasion dynamics are paramount to personalizing patient care and improving outcomes. We developed a novel quantitative approach to infer such dynamics characteristics from a single biopsy taken at patient diagnosis. This result represents a fundamental step toward integrating quantitative methods into clinical decision-making to improve treatment responses and outcomes.

### Example Methods

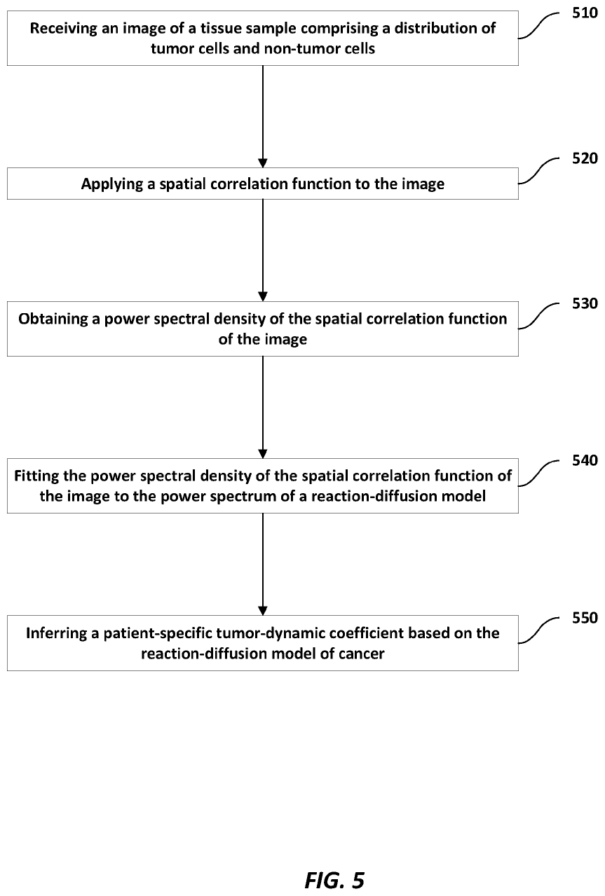

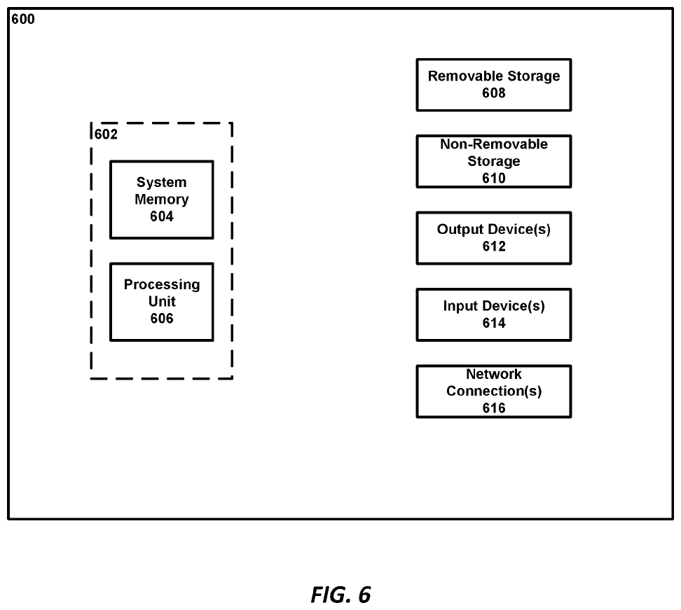

FIG. 5 is a flowchart of an example method for determining patient-specific tumor dynamics according to implementations described herein. It should be understood that the computer-implemented methods described herein can be performed using a computing device (e.g., computing device 600 of FIG. 6). According to the method described herein, a tissue sample (and optionally a single tissue sample) can be used to calibrate a reaction-diffusion model of cancer and therefore be used to simulate individual patient growth curves and treatment response dynamics.

At step 510, the method includes receiving an image of a tissue sample comprising a distribution of tumor cells and non-tumor cells. In the Examples described below, the image is an immunofluorescence stained slide image. Such an image is illustrated in FIG. 13 and described in the Examples below. It should be understood that immunofluorescence staining is provided only as an example. This disclosure contemplates that the image may be an immunohistochemistry (IHC) stained slide image, a hematoxylin & eosin (H&E) stained slide image, or other stained slide images. Alternatively, the image may be captured by a different imaging modality.

Additionally, in some implementations, the tissue sample is a pre-treatment sample. Alternatively or additionally, in some implementations, the tissue sample is a post-treatment sample. Optionally, in some implementations, only a single image is required for the methods described below. In other words, the patient-specific tumor-dynamic coefficient can be derived from the reaction-diffusion model of cancer using the single image using the methods described below. Deriving patient-specific tumor-dynamic coefficients according to conventional technologies requires multiple images (i.e. images acquired at different time points).

Additionally, in some implementations, the cancer is breast cancer. It should be understood that breast cancer is provided only as an example. This disclosure contemplate that the cancer is another type, for example, head and neck cancer or prostate cancer.

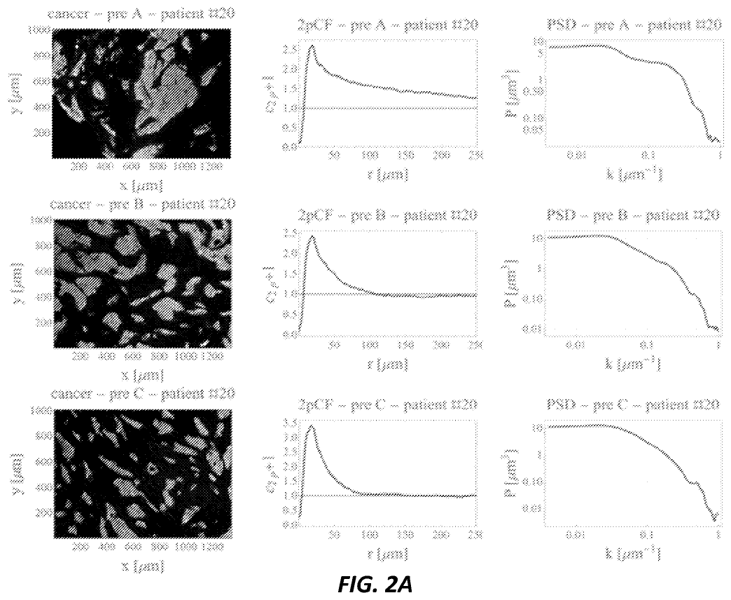

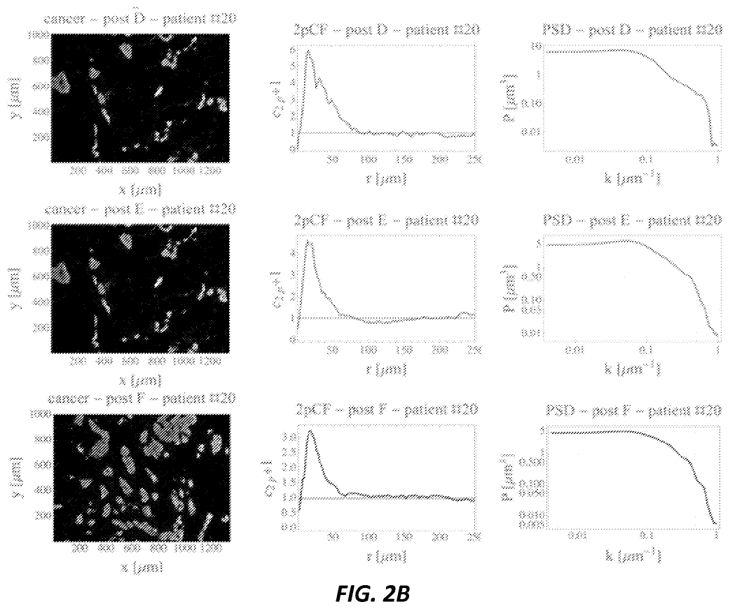

At step 520, the method includes applying a spatial correlation function to the image. As described in the Examples below, the middle column in FIGS. 2A-2B illustrate a 2-point correlation function of the image. In some implementations, the spatial correlation function is a 2-point correlation function. In some implementations, the spatial correlation function is a 2-point cross-correlation function. It should be understood that the 2-point correlation function and 2-point cross-correlation function are provided only as examples.

At step 530, the method includes obtaining a power spectral density (PSD) of the spatial correlation function of the image. As described in the Examples below, the right column in FIGS. 2A-2B illustrate the PSD of the 2-point correlation function of the image. Optionally, the power spectral density is obtained by applying a Fourier transform to the spatial correlation function of the image. This disclosure contemplates that this can be accomplished using the Discrete Fourier Transform (DFT) algorithm or the Fast Fourier Transform (FFT) algorithm. It should be understood that DFT and FFT algorithms are provided only as examples. This disclosure contemplates using any algorithm that transforms the spatial correlation function of the image (e.g., time-domain image) into its frequency components.

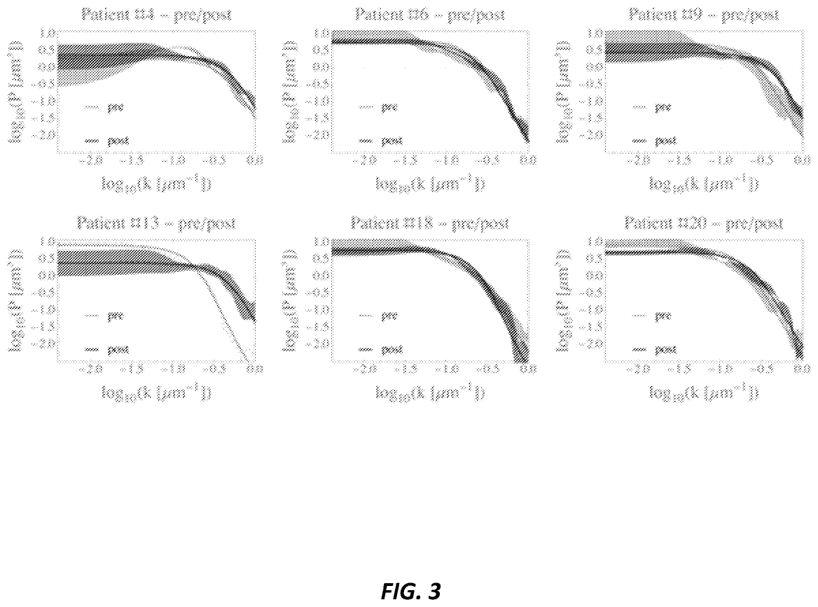

At step 540, the method includes fitting the power spectral density of the spatial correlation function of the image to the power spectrum of a reaction-diffusion model. As described in the Examples below, the solid lines in FIG. 3 illustrate fitting the PSD to the power spectrum of a reaction-diffusion model.

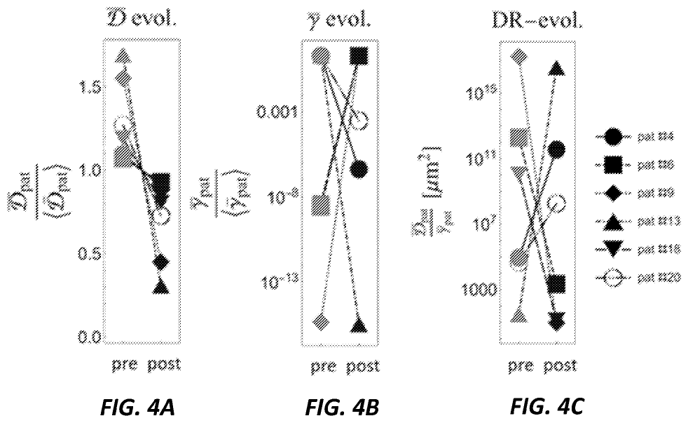

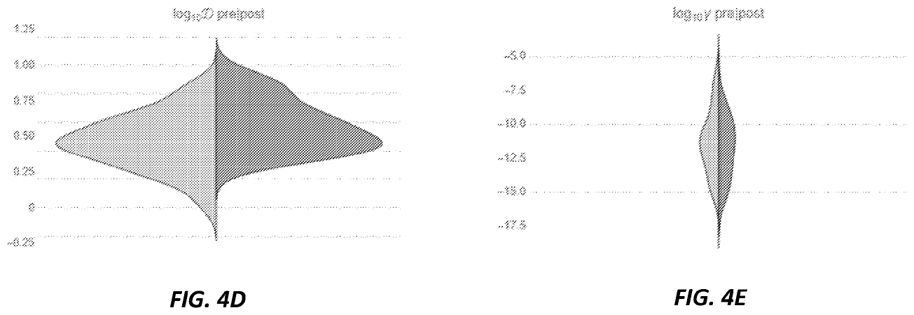

At step 550, the method includes inferring a patient-specific tumor-dynamic coefficient based on the reaction-diffusion model of cancer. As described in the Examples below, FIGS. 4A-4C illustrate inferring patient-specific tumor-dynamic coefficients based on the reaction-diffusion model. The patient-specific tumor-dynamic coefficient is a growth rate or a diffusion rate. In some implementations, the patient-specific tumor-dynamic coefficient is a growth rate. In some implementations, the patient-specific tumor-dynamic coefficient is a diffusion rate. In some implementations, the patient-specific tumor-dynamic coefficient is both a growth rate and a diffusion rate.

Optionally, the method optionally further includes simulating a tumor's response to treatment using the patient-specific tumor-dynamic coefficient.

An example method of treating cancer in a subject is described herein. The method includes determining patient-specific tumor dynamics for the subject according to the techniques described herein (see e.g., the operations of FIG. 5); and guiding a treatment regimen for the subject using the patient-specific tumor-dynamic coefficient. Tumor dynamics with a high growth rate are very susceptible to cytotoxic therapy such as radiation, chemotherapy, targeted therapy. Additionally, tumors with high diffusion rate may be more diffusive and higher risk of locoregional invasion and would need a more systemic approach, which can be facilitated using the techniques described herein.

Optionally, the step of guiding the treatment regimen for the subject includes increasing or decreasing an amount or frequency of treatment administered to the subject. Alternatively or additionally, the treatment regimen is optionally one or more of chemotherapy, immunotherapy, or radiotherapy. Optionally, the treatment regimen is a combination therapy. Optionally, the step of guiding the treatment regimen for the subject may include selecting one or more of chemotherapy, immunotherapy, or radiotherapy based on the patient-specific tumor-dynamic coefficient.

Alternatively or additionally, the method optionally further includes administering the treatment regimen to the subject.

Another example computer-implemented method for determining patient-specific tumor dynamics is described herein. It should be understood that the computer-implemented methods described herein can be performed using a computing device (e.g., computing device 600 of FIG. 6). The method includes receiving a single image of a tissue sample that includes tumor cells; analyzing the single image; and deriving patient-specific tumor-dynamic information based on the analysis.

Yet another computer-implemented method for determining patient-specific tumor dynamics is described herein. It should be understood that the computer-implemented methods described herein can be performed using a computing device (e.g., computing device 600 of FIG. 6). The method includes receiving a medical image of tissue that includes a distribution of tumor cells and non-tumor cells; applying a spatial correlation function to the medical image; and obtaining a power spectral density of the spatial correlation function of the medical image. The method also includes fitting the power spectral density of the spatial correlation function of the medical image to the power spectrum of a reaction-diffusion model; and inferring a patient-specific tumor-dynamic coefficient based on the reaction-diffusion model of cancer.

Additionally, the medical image is a magnetic resonance image (MRI), positron emission tomography (PET) image, or a computer tomography (CT) image.

An example computer-implemented method for classifying tumor cells is also described herein. It should be understood that the computer-implemented methods described herein can be performed using a computing device (e.g., computing device 600 of FIG. 6). The method includes receiving an image of a tissue sample that includes a distribution of tumor cells and non-tumor cells; applying a spatial correlation function to the image; and classifying one or more cells in the image as a tumor or non-tumor cell using the spatial correlation function.

### Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Breast Cancer Reaction-Diffusion from Spectral-Spatial Analysis in Immunohistochemistry

### Results

### Breast Tumor Detection by 2-Point Correlation Function.

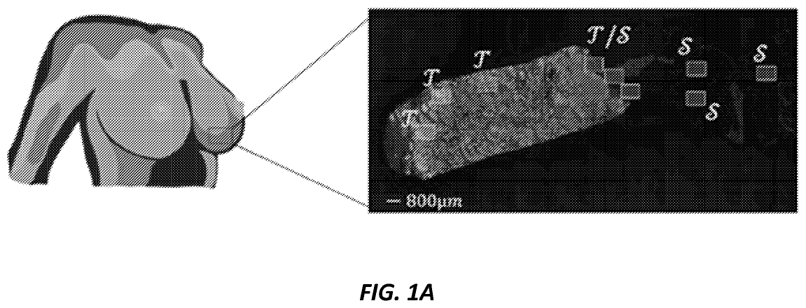

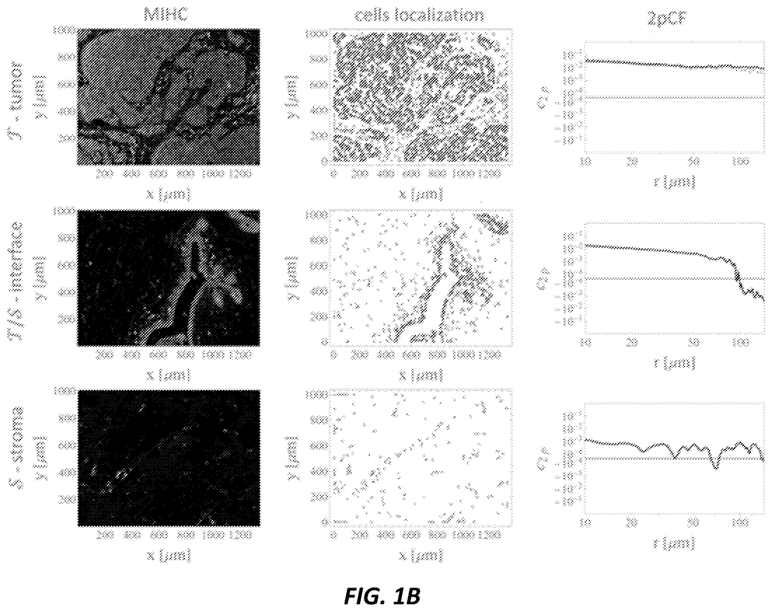

We analyze patient histopathology-stained slides (FIG. 1A) spatial cell distributions with the 2-point correlation function (hereafter c₂ₚ, or 2pCF). This statistical tool describes how cancer cell density varies as a function of the distance r from any cell (33). Three Regions of Interest (ROI) are selected, including tumor ( [symbol] ), stroma distant to the tumor ( [symbol] ), tumor and stroma interface ( [symbol] - [symbol] , FIG. 1B).

The combined tumor and stroma cell distribution in a [symbol] -ROI proposes a characteristic monomial distribution signature. Analysis of the spatial distribution of tumor cells alone follows a similar linear relationship in the bi-logarithmic plot. The correlation is mainly positive in both cases, meaning an architectural aggregation of the cells in the ROI exceeds the Poisson distribution (i.e., randomness). By contrast, cell clustering in stroma-dominated ROI, [symbol] , presents a more rapid loss of positive correlation to favor an irregularly oscillatory 2pCF profile at about one order of magnitude smaller values than for T-ROI. The 2pCF in [symbol] -ROI approximates c₂ₚ≅0, equivalent to a random cellular distribution, without any evident characteristic signature or regularity in the sample analyzed.

Finally, the bi-logarithmic plot for intermediate [symbol] - [symbol] ROI presents a monomial behavior at low-medium r, similar to what is evidenced for T ROI. Still, it shows an abrupt drop that characterizes it toward largely negative c₂ₚ at asymptotically larger r (less clustered than a random distribution).

### Radiation Therapy Induces Changes in Cancer Cell Distribution Morphology.

We analyze the spatial distribution of cancer cells in the different [symbol] ROI before (FIG. 2A) and after (FIG. 2B) SABR (Stereotactic Ablative Radiotherapy). We investigate c₂ₚ+1 to evaluate only positive values of the 2pCF. The Poisson-process threshold moves from 0 to 1, with c₂ₚ>1 representing spatial correlation or cell clustering, and c₂ₚ<1 representing anticorrelation for [symbol] cell distribution alone. Cancer cells in three different [symbol] ROI both pre- and post-treatment (referred to as A, B, and C in FIG. 2A, as well as D, E, and F in FIG. 2B, respectively) strongly cluster in close neighborhoods (r<50 μm) but lose positive correlation with a different characteristic slope as they approach Poisson randomness at more considerable distances (r>50 μm; FIGS. 2A-2B, second column). Furthermore, we consider the 2pCF Fourier transform, the power spectral density (PSD), to capture better the multiscale contribution to the cancer architectural organization (tracked by wavenumbers, k) across the resection area. The characteristic “white-noise” plateau at small wavenumbers is followed after a characteristic wavenumber value where the PSD shape bends downward (sometimes referred to as correlation-length) by the drop of the PSD at higher k (FIGS. 2A-2B, third column).

### Histopathology Slides Power Spectrum Analysis Calibrates Reaction-Diffusion Equation Parameters.

Patient tissues provide a single-time snapshot of the complex evolution of the cellular density in different spatial locations inside the resection area (FIGS. 1A-B). The exact location of each cancer cell and cancer cell conglomerates gives insights into the tumor proliferation and invasion dynamics. The reaction-diffusion (RD) equation represents a family of models relating the density of cancer cells in a given position at a given time as a function of the proliferation rate γ (per unit time) and a diffusion coefficient [symbol] (area per unit time). We derive the analytical formulation of the PSD for the homogeneous-isotropic RD equation and solve the RD parameter inference problem by interpreting the observed PSD of the histopathology slides with the PSD derived from the RD equation. The PSD of the RD equation closely captures the PSD of the pre- and post-treatment tissues (R²>0.9; FIG. 3).

Furthermore, the RD framework disentangles the different contributions between proliferation and diffusion by systematic differences in pre and post-treatment PSD. A direct comparison of pre-and-post-SABR RD parameters demonstrates a reduction in the patient-specific diffusion parameter, [symbol] ₚₐₜ, for each patient normalized by the average patient-specific diffusion parameter, [symbol] (i.e., across all [symbol] ROI pre and post-SABR, FIG. 4A). However, the normalized patient-specific growth rate parameter showed a dichotomy of responses for the γₚₐₜ\sqrt{γₚₐₜ}: increasing for some and decreasing for other patients (FIG. 4B). In FIG. 4C, the ratio of the coefficients (not averaged) is shown to track the cumulative effect of disease diffusion and proliferation: SBRT may induce an increase in the ratio with expected tumor growth (patients #6, #9, #18), while a decreasing trend suggests likely post-SBRT tumor regression (patient #4, #13, #20). FIGS. 4D and 4E illustrate the probability distribution function for diffusion coefficient ( [symbol] ) and reaction rates (γ), respectively, for the whole set of 53 patients.

### Discussion

Cancer is a complex adaptive dynamic system that evolves at multiple scales; therefore, a trans-levels technique that captures the characteristics of several scales (or equivalently over several wavenumbers) is of fundamental importance. Here we presented the first step in developing such an interpretative methodology to capture the dominant scale lengths that drive breast cancer architectures before and after stereotactic ablative radiotherapy. Herein, we explore the potential of the cancer-cell architecture-defragmentation analysis and interpret it as a growth and tissue invasion process through the RD equation. The two-point correlation function connects spatial architecture and tumor growth and diffusion. The power spectral density is obtained directly from patient tissue immunofluorescence histopathology slides to calibrate the parameters of the RD equation. Such an approach requires a few theoretical and biological hypotheses, particularly the “fair representation” hypothesis that histopathology slides are reasonably representative of the clustering properties of the total tumor population. While the tumor's loco-regional properties may be well captured in an individual drop, the overall tumor is highly heterogeneous, and the cancer cell population's local characteristics might depend on the micro-environment. We assumed that the sampled ROI is representative of the overall patient disease.

Furthermore, we adopted a “strong” version of the fair-representation hypothesis: we assumed that the analysis presented represents the characteristic of the disease type. The fair-representation hypothesis then permits us to generalize the consideration by comparing different patient biopsies and different patients, empowering our considerations. I.e., we assume that we are studying not one patient breast cancer but that we can extend our consideration to all breast cancers. Therefore, to achieve the goals of the present work, we took the validity of histopathology spatial characterization worked out with the 2pCF as fairly representing the nature of the breast cancer and considered average trends over the sample of patients available to us. This stretch will be tested when additional, more extended databases of breast cancer histopathology become available. While potentially conceived as a limitation; instead, this approach offers the possibility to control on a cell by cell basis the connection between spatial statistical tools, the two-point correlation function, and the corresponding power spectrum.

The second strong hypothesis is found in the choice of the interpretative framework: the RD equation. While the 2pCF and the corresponding PSD from the dataset are virtually free of assumption, the RD framework was not derived from the data but chosen due to its simplicity. No reason a priori justifies the choice and form of the equation if not the practical analytical tractability, especially in Fourier space. While more physically involved approaches exist, such as Navier Stokes approaches, especially in the glioma literature (see introductive references), numerical investigation of integrative instruments and the power spectral density (which easily captures several orders of magnitude in wavelength) would involve multiscale physical parametrization that is not justified by the resolution and quantity of the available data. Thus, the chosen approach aligns with the clinical dataset and moves toward a predictive quantitative oncology framework. The clinical data set only provided matched pre- and post-SBRT tissue samples for six patients with comparable outcomes for quantitative analysis. Therefore, the focus of this work is the development of breast cancer patient-specific reaction-diffusion dynamics from spectral analysis of immunohistochemistry slides. Future work will include analysis of such derived dynamics to predict complete pathological responses, relapse-free, and overall survival to help guide clinical decision-making (34).

Finally, this disclosure contemplates that 2pCF analyses can be used to build a robust classifier to detect the presence of cancer architectures in selected biopsy ROI. For example, a method for classifying for classifying tumor cells may include receiving an image of a tissue sample that includes a distribution of tumor cells and non-tumor cells; applying a spatial correlation function to the image; and classifying one or more cells in the image as a tumor or non-tumor cell using the spatial correlation function.

### Materials and Methods

### Patient Data.

A total of Nₚₐₜ=53 patients have been accrued to a Phase II Study of preoperative SABR with three times 9.5 Gy for early-stage breast cancer (NCT03137693). A 5 μm thick unstained tumor slide from formalin-fixed paraffin-embedded tumor block was acquired from biopsy and post-SABR and analyzed by the Moffitt Cancer Center tissue core and digital imaging laboratory. Tissues were stained with multiplex immunofluorescence to evaluate the infiltration of antigen-presenting cells, T-cells, NK cells, B-cells, and tumor cells using different combinations of CD3 and CD4 CD8, Foxp3, PD-1, CD68, PanCytoKeratin (PCK). For six patients, we have both pre- and post-treatment data. We focus on this subset of patients in the main text, leaving to the Supplement (see Example 1) the investigation of patients with either pre- or post-treatment alone.

2-Point Correlation Function (2pCF).

We implement the 2-point correlation function as a spatial statistic estimator for our project (35-39). The spatial n-point autocorrelation function cₙₚ is defined as the probability of finding n cells at coordinates x₁, x₂, . . . , xₙ in a suitably defined reference frame S(O,x) centered in O with xᵢ={x₁, x₂, x₃}ᵢ as spatial coordinates for the i=1, 2, 3, . . . , n. If we refer to the cell type j, j∈T with T set of possible cell types T={ [symbol] , [symbol] , CD3⁺, CD4⁺, CD8⁺, FOXP3, . . . }, where [symbol] -tumor cell population, [symbol] -stroma cell population, and so forth, then for the generic set type j in our histopathology-stained slide, we can formalize

$${c}_{np}^{( j )} as {c}_{np}^{( j )} = {〈 δ ( {x}_{1} ) δ ( {x}_{2} ) , … , δ ( {x}_{n} ) 〉}^{( j )} .$$

Note how the significant differences between npCf and cooccurrence (Haralick et al. 1973) are, aside from normalization factors, the pixeled/grid-free definition of the npCf and the possibility to capture both geometry and statistical nature of the images analyzed without grey-levels definition.

We focus on the 2pCF of the cancer population. We drop index j and refer to the 2pCF as the auto-correlation function. Furthermore, because of the connection we are going to develop in the next section with the continuous RD equation, we are going to implement for the 2pCF the following definition. We note that the definition of the correlation function (or of the covariance function, or its relation with the Ripley's K function) is not universal across the different disciplines and textbooks: the subtraction of the average density or any uncorrelated average product of 2 random variables is a matter of convention that, therefore we fix here with this definition.

$${c}_{2 p} ( r , τ ) = \frac{〈 ( ρ ( x + r , t + τ ) - 〈 ρ 〉 ) ( ρ ( x , t ) - 〈 ρ 〉 ) 〉}{{〈 ρ 〉}^{2}} , \qquad ( 3.1 )$$

with [symbol] =n the average number of cells for volume unit, and r=∥r∥≡∥x₁−x₂∥, τ a time interval. Because we work with a single breast cancer histopathology slide, we cannot sample the same tissue section twice, and we cannot consider the time dependence of the autocorrelation. Therefore we simplify Eq. (3.1) to:

$$〈 ρ ( x + r ) ρ ( x ) 〉 = {n}^{2} ( 1 + {c}_{2 p} ( r ) ) . \qquad ( 3.2 )$$

The interpretation of this type of correlation function is simple: if the probability of finding a cell in the volume dV is dP=ndV with n the average number of cells in the finite volume spanned by the histopathology slide, then (N)=nV and the joined probability of finding two cells say cell 1 and cell 2, at a given distance r, is

$$d P = {n}^{2} d {V}_{1} d {V}_{2} ( 1 + {c}_{2 p} ( r ) ) , \qquad ( 3.3 )$$

which gives us also a concept interpretative key: If the cell distribution is a 3D random Poisson point process, the probability of finding cells in dV₁ and dV₂ are independent, in this case c₂ₚ=0, if the cell positions are correlated c₂ₚ>0, if the positions are anticorrelated c₂ₚ∈[−1,0[.

Note that to properly account for the 3D overlapping of cells in the spatial analysis of the 2pCF, we couped Eq. (3.2) with several kernels, boundary conditions, and bandwidth, always finding convergence (but not the coincidence) of results.

### Power Spectrum of the Fluctuations.

For a continuous function, the Fourier transform of the autocorrelation function is the power spectrum. We only review the analogous for a 3D random point process we use in the following, focusing on and developing only the equations of interest (40). The Fourier transform for the distribution of cells with density ρ(x,t)=( [symbol] V)⁻¹Eᵢδ(x−xᵢ(t)) is

$${δ}_{k} = {( nV )}^{- 1} {Σ}_{i} {e}^{ι k \cdot {x}_{i}} , \qquad ( 3.4 )$$

with wavenumber k=∥k∥, k={kₓ,k_{y},k_{z}}, where the barycenter of the i^{th} cell is located at xᵢ and e^{ιk·x} is periodic in V (ι is the complex imaginary units, “·” the inner product). We slice the volume V (we refer to the slide and its volume with the same symbol V without loss of generality) into infinitesimal cells with unitary function 1_{dV} (1_{dV}=1 if the cell is in the volume V, or 1_{dV}=0 if it is not). Therefore Eq. (3.4) is equivalent to

$${δ}_{k} = {( n V )}^{- 1} {Σ}_{i} {1}_{dV} {e}^{ι k \cdot {x}_{i}} . \qquad ( 3.5 )$$

Hence, the averaged two cells contribution to the spectrum of wavelengths, say cell 1 in dV₁ and cell 2 in dV₂, reads:

$${( nV )}^{2} 〈 {δ}_{k} {δ}_{- {k}^{′}} 〉 = Σ 〈 {1}_{1}^{2} 〉 {e}^{ι ( k - {k}^{′} ) \cdot {x}_{1}} + Σ 〈 {1}_{1} {1}_{2} 〉 {e}^{ι ( k \cdot {x}_{1} - {k}^{′} \cdot {x}_{2} )} = n {\int }_{{ℝ}^{3}} {dVe}^{ι ( k - {k}^{′} ) \cdot {x}_{1}} + {n}^{2} {\int }_{{ℝ}^{3}} {dV}_{1} {dV}_{2} ( 1 + {c}_{2 p} ) {e}^{ι ( k \cdot {x}_{12} + ( k - {k}^{′} ) \cdot {x}_{2} )} , \qquad ( 3.6 )$$

because [symbol] 1₁1₂ [symbol] =n²dV₁dV₂(1+c₂ₚ) as results of considering Eq. (3.3) (with x₁₂ Euclidean distance between cell 1 and 2). Because Fourier components belonging to different k are statistically independent, the integrals in the previous vanishes if k≠k′, while for k=k′ yields, as a well known results, the spectrum (the power spectral density, PSD) of the cell distribution:

$$P ( k ) ≡ 〈 {\vert {δ}_{k} \vert}^{2} 〉 = {\int }_{{ℝ}^{3}} \frac{{d}^{3} x}{V} {c}_{2 p} ( r ) {e}^{ι k \cdot x} + \frac{1}{nV} \qquad ( 3.7 )$$

for k≠0, and

$$〈 {\vert {δ}_{0} \vert}^{2} 〉 = {\int }_{{ℝ}^{3}} \frac{{d}^{3} r}{V} {c}_{2 p} + \frac{1}{nV}$$

otherwise. The power spectrum measures the mean number of neighbors more than a random Poisson distribution within a distance of ˜k⁻¹ of a randomly chosen cell. This classical result (e.g., (41)) is often coupled with filter-design windows functions commonly implemented in the power spectrum determination to optimize its analytical treatment (Dirichlet, Hamming, Blackman, etc.) that we also tested (see Example 1) with consistent results. In this paper, we will stick to the derivation presented above. Nuttall-softening length (42) will be preferred if necessary.
2-Point Correlation Function from the Reaction-Diffusion Equation.

Diffusion of a cluster of cells with density ρ=ρ(r,t) at position r and at time t is described by the combination of the continuity equation without source and sink terms as ∂ₜρ+∇·φ=0 (with ∂ being the partial derivative and ∇ the gradient operator) and Fick's first law of proportionality between flux φ and density gradient ∇ρ, φ=− [symbol] ·∇ρ, to get (for a generic anisotropic flow) ∂ₜρ=−∇. ( [symbol] Vρ) where [symbol] is a diffusion coefficient matrix. In a 3D system of reference (SoR) S(O,r={x,y,z}), with r²=x²+y²+z² being the squared radial direction from an arbitrary origin O, we can write the diffusion equation for the homogeneous and isotropic case as ∂ₜρ= [symbol] ∇²ρ, where ∇² is the D'Albertian operator. Green functions offer a natural solution for cell-like source points, even in the presence of an explicit exponential growth factor γ, i.e., ∂ₜρ= [symbol] ∇²ρ+γρ, in the form:

$$ρ ( r , t ) = \frac{{ρ}_{0}}{{( 4 π𝒟 t )}^{\frac{3}{2}}} {e}^{- \frac{{‖ r ‖}^{2}}{4 𝒟 t} + γ t} , \qquad ( 3.8 )$$

with ∥*∥=∥*∥₂ Euclidean norm. Note how this approach follows exponential tumor growth without carrying capacity constraints, and we will comment on this limitation later. For now, we focus on tumor RD statistics as an average over the tumor ROI, [symbol] . We assume that the cells are spread over a small volume d³r so that, as long as this volume is asymptotically smaller than the cell migration movement per unit time, say dr<O(√ [symbol] dt), we can generalize the previous equation as

$$ρ ( r , t ) = {ρ}_{0} {d}^{3} {r ( 4 π𝒟 t )}^{- \frac{3}{2}} {e}^{- \frac{{‖ r ‖}^{2}}{4 𝒟 t} + γ t} .$$

In the case of n different cells

$$ρ ( r , t ) = {( 4 π𝒟 t )}^{- \frac{3}{2}} {ρ}_{0 i} {e}^{- \frac{{‖ {r}_{i} ‖}^{2}}{4 𝒟 t} + γ t} {d}^{3} r$$

(where Einstein's muted index convention is assumed) or, in the passage to the continuous limit, we get the classical heat-equation result that we are going to exploit here for our RD problem:

$$ρ = {\int }_{{ℝ}^{3}} {d}^{3} {r}^{′} {ρ}_{0} ( {r}^{′} ) {( 𝒟 t )}^{- \frac{3}{2}} {e}^{- \frac{{‖ r - {r}^{′} ‖}^{2}}{4 𝒟 t} + γ t} = {ρ}_{0} * {f}_{R D P S} . \qquad ( 3.9 )$$

The last term in the equation is the one of interest here. The cell concentration at the generic time t is the convolution, denoted here by “*”, of the initial concentration with a Gaussian, that by borrowing the terminology from Astronomy, we call RD point-spread-function ƒ_{RDPS}. Here cell concentration at +∞ is assumed to be null. Furthermore, by assuming constant temperature and pressure in the tissue sample, we justify [symbol] =cnst. and elaborate on the ƒ_{RDPS} as the function that carries the temporal dependence.

Under the assumption of the validity of the fair-representation hypothesis, we assume a mean-field solution for the RD equation over the resection area from which the histopathology slides come. By performing this Gibbs average, we require that the area over which the average is performed is large enough to contain a sufficient number of cells to perform statistical analyses but small enough to neglect large-scale gradients (i.e., [symbol] =cnst holds reasonably well). In this case, from Eq. (3.9) we obtain:

$$〈 ρ 〉 = 〈 {ρ}_{0} 〉 * 〈 {f}_{R D P S} 〉 , \qquad ( 3.1 )$$

where we define the average over the time of the RD spread function as:

$${f}_{R D} ≡ 〈 {f}_{R D P S} 〉 = \frac{1}{Δ t} {\int }_{0}^{Δ t} {f}_{R D P S} , \qquad ( 3.11 )$$

with Δt sufficiently small that will indeed account for the non-equilibrium dynamics of diffusion and proliferation required by the average in ρ₀. The drawback of this approach is that the same patient presents different diffusion coefficients D and normalization densities ρ₀ in distinct resection areas instead of having continuous functions for [symbol] = [symbol] (x,t) and growth rate γ=γ(x,t) dependent on spatial position. Furthermore, stochastic correlation is different from [symbol] , and [symbol] area (FIG. 1B), and we expect the theory to break down at the [symbol] - [symbol] interface. Therefore, we are forced to solve the RD equation only locally in [symbol] . Finally, by considering the non-normalized autocorrelation of the average cell concentration [symbol] , we write

$${c}_{2 p} = ( ( {ρ}_{0} - ( {ρ}_{0} 〉 ) * ( {ρ}_{0} - 〈 {ρ}_{0} 〉 ) ) * ( {f}_{R D} * {f}_{R D} ) = {σ}_{ρ}^{2} ( {f}_{R D} * {f}_{R D} ) , \qquad ( 3.12 )$$

where the initial density is non-correlated, and the correlation function reduces to its variance σ_{ρ}. The biological implication is that diffusing cells enhances cell proliferation—a visualization of loss of space and contact inhibition. Therefore, these reactions are stochastically correlated, and Eq. (3.12) assumes that cell diffusion is the only mechanism by which cells become spatially correlated. The autocorrelation of ƒ_{RD} captures this spatial correlation thusly not contradicting Eq. (3.10) as ergodicity does not imply mixing.

Power Spectrum from Reaction-Diffusion Equation.

The RD equation's analytical solution is unavailable for arbitrary reaction terms; therefore, it is easier to work with power spectral densities than 2-point correlation functions to connect cancer diffusion with its spatial distribution. Performing Eq. (3.11) on Eq. (3.8), we obtain ƒ_{DR} as

$${f}_{R D} = \frac{{e}^{ι ‖ x ‖ \sqrt{\frac{γ}{𝒟}}}}{8 π𝒟 ‖ x ‖} erfc ( \frac{‖ x ‖}{2 \sqrt{𝒟 Δ t}} + ι \sqrt{γ Δ t} ) + \frac{{e}^{- ι ‖ x ‖ \sqrt{\frac{γ}{𝒟}}}}{8 π𝒟 ‖ x ‖} erfc ( \frac{‖ x ‖}{2 \sqrt{𝒟 Δ t}} - ι \sqrt{γ Δ t} ) \qquad ( 3.13 )$$

where erfc(*) is the complementary error function. Finally, considering Eq. (3.12), Eq. (3.13) together with the definition of power spectral density, we obtain

$$P ( k ) ≡ {\vert {\int }_{{ℝ}^{3}} {d}^{3} x {f}_{R D} {e}^{ι k \cdot x} \vert}^{2} = {{P}_{0}^{2} ( \frac{{e}^{( γ - {‖ k ‖}^{2} 𝒟 ) Δ t} - 1}{Δ t ( γ - {‖ k ‖}^{2} 𝒟 )} )}^{2} \qquad ( 3.14 )$$

where for easier comparison with Eq. (3.7) we normalized with P₀ to the zero-wavelength by computing ∫_{R}_{³}P(k)d³k to obtain our final result.

We finally perform an inference exercise of the model Eq. (3.7) to the PSD data with the same Bayesian/Frequentists techniques presented in (43) or advocated in (34). Any other inference approach is equally valid.

### Example Computing Device

It should be appreciated that the logical operations described herein with respect to the various figures may be implemented (1) as a sequence of computer implemented acts or program modules (i.e., software) running on a computing device (e.g., the computing device described in FIG. 6), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 6, an example computing device 600 upon which the methods described herein may be implemented is illustrated. It should be understood that the example computing device 600 is only one example of a suitable computing environment upon which the methods described herein may be implemented. Optionally, the computing device 600 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 600 typically includes at least one processing unit 606 and system memory 604. Depending on the exact configuration and type of computing device, system memory 604 may be volatile (such as random access memory (RAM)), non-volatile (such as read-only memory (ROM), flash memory, etc.), or some combination of the two. This most basic configuration is illustrated in FIG. 6 by dashed line 602. The processing unit 606 may be a standard programmable processor that performs arithmetic and logic operations necessary for operation of the computing device 600. The computing device 600 may also include a bus or other communication mechanism for communicating information among various components of the computing device 600.

Computing device 600 may have additional features/functionality. For example, computing device 600 may include additional storage such as removable storage 608 and non-removable storage 610 including, but not limited to, magnetic or optical disks or tapes. Computing device 600 may also contain network connection(s) 616 that allow the device to communicate with other devices. Computing device 600 may also have input device(s) 614 such as a keyboard, mouse, touch screen, etc. Output device(s) 612 such as a display, speakers, printer, etc. may also be included. The additional devices may be connected to the bus in order to facilitate communication of data among the components of the computing device 600. All these devices are well known in the art and need not be discussed at length here.

The processing unit 606 may be configured to execute program code encoded in tangible, computer-readable media. Tangible, computer-readable media refers to any media that is capable of providing data that causes the computing device 600 (i.e., a machine) to operate in a particular fashion. Various computer-readable media may be utilized to provide instructions to the processing unit 606 for execution. Example tangible, computer-readable media may include, but is not limited to, volatile media, non-volatile media, removable media and non-removable media implemented in any method or technology for storage of information such as computer readable instructions, data structures, program modules or other data. System memory 604, removable storage 608, and non-removable storage 610 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 606 may execute program code stored in the system memory 604. For example, the bus may carry data to the system memory 604, from which the processing unit 606 receives and executes instructions. The data received by the system memory 604 may optionally be stored on the removable storage 608 or the non-removable storage 610 before or after execution by the processing unit 606.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### REFERENCES

1. H. Hatzikirou, et al., Integrative physical oncology. WIREs Systems Biology and Medicine 4, 1-14 (2012).
2. R. A. Gatenby, E. T. Gawlinski, A reaction-diffusion model of cancer invasion. Cancer Res 56, 5745-5753 (1996).
3. K. R. Swanson, E. C. Alvord, J. D. Murray, A quantitative model for differential motility of gliomas in grey and white matter. Cell Prolif 33, 317-329 (2000).
4. K. R. Swanson, et al., A Quantitative Model for the Dynamics of Serum Prostate-Specific Antigen as a Marker for Cancerous Growth: An Explanation for a Medical Anomaly. The American Journal of Pathology 158, 2195-2199 (2001).
5. K. R. Swanson, E. C. Alvord, J. D. Murray, Virtual brain tumours (gliomas) enhance the reality of medical imaging and highlight inadequacies of current therapy. Br J Cancer 86, 14-18 (2002).
6. K. R. Swanson, C. Bridge, J. D. Murray, E. C. Alvord, Virtual and real brain tumors: using mathematical modeling to quantify glioma growth and invasion. J Neurol Sci 216, 1-10 (2003).
7. Z. Szymanska, Urbanski, Marciniak-Czochra, Mathematical modelling of the influence of heat shock proteins on cancer invasion of tissue | SpringerLink (2009) (May 5, 2021).
8. A. Martinez-Gonzalez, L. Perez Romasanta, V. Perez-Garcia M., Hypoxic Cell Waves Around Necrotic Cores in Glioblastoma: A Biomathematical Model and Its Therapeutic Implications | SpringerLink (2012) (May 10, 2021).
9. C. H. Wang, et al., Prognostic Significance of Growth Kinetics in Newly Diagnosed Glioblastomas Revealed by Combining Serial Imaging with a Novel Biomathematical Model. Cancer Res 69, 9133-9140 (2009).
10. D. Pally, D. Pramanik, R. Bhat, An Interplay Between Reaction-Diffusion and Cell-Matrix Adhesion Regulates Multiscale Invasion in Early Breast Carcinomatosis. Front. Physiol. 10 (2019).
11. M. U. Bogdariska, et al., A mathematical model describes the malignant transformation of low grade gliomas: Prognostic implications. PLOS ONE 12, e0179999 (2017).
12. A. Henares-Molina, et al., Non-standard radiotherapy fractionations delay the time to malignant transformation of low-grade gliomas. PLoS One 12, e0178552 (2017).
13. P. Gerlee, S. Nelander, The impact of phenotypic switching on glioblastoma growth and invasion. PLoS Comput Biol 8, e1002556 (2012).
14. R. L. Klank, S. S. Rosenfeld, D. J. Odde, A Brownian dynamics tumor progression simulator with application to glioblastoma. Converg. Sci. Phys. Oncol. 4, 015001 (2018).
15. J. Khetan, M. Shahinuzzaman, S. Barua, D. Barua, Quantitative Analysis of the Correlation between Cell Size and Cellular Uptake of Particles. Biophys J 116, 347-359 (2019).
16. S. Angeli, K. E. Emblem, P. Due-Tonnessen, T. Stylianopoulos, Towards patient-specific modeling of brain tumor growth and formation of secondary nodes guided by DTI-MRI. NeuroImage: Clinical 20, 664-673 (2018).
17. J. A. Weis, et al., A mechanically coupled reaction-diffusion model for predicting the response of breast tumors to neoadjuvant chemotherapy. Phys. Med. Biol. 58, 5851-5866 (2013).
18. J. A. Weis, et al., Predicting the Response of Breast Cancer to Neoadjuvant Therapy Using a Mechanically Coupled Reaction-Diffusion Model. Cancer Res 75, 4697-4707 (2015).
19. J. A. Weis, M. I. Miga, T. E. Yankeelov, Three-dimensional image-based mechanical modeling for predicting the response of breast cancer to neoadjuvant therapy. Computer Methods in Applied Mechanics and Engineering 314, 494-512 (2017).
20. T. Roque, et al., A DCE-MRI Driven 3-D Reaction-Diffusion Model of Solid Tumor Growth. IEEE Trans Med Imaging 37, 724-732 (2018).
21. A. M. Jarrett, et al., Incorporating drug delivery into an imaging-driven, mechanics-coupled reaction diffusion model for predicting the response of breast cancer to neoadjuvant chemotherapy: theory and preliminary clinical results. Phys Med Biol 63, 105015 (2018).
22. A. Mang, S. Bakas, S. Subramanian, C. Davatzikos, G. Biros, Integrated Biophysical Modeling and Image Analysis: Application to Neuro-Oncology. Annu Rev Biomed Eng 22, 309-341 (2020).
23. A. Roniotis, K. Marias, V. Sakkalis, G. C. Manikis, M. Zervakis, Simulating radiotherapy effect in high-grade glioma by using diffusive modeling and brain atlases. J Biomed Biotechnol 2012, 715812 (2012).
24. C. H. Holdsworth, et al., Adaptive IMRT using a multiobjective evolutionary algorithm integrated with a diffusion-invasion model of glioblastoma. Phys. Med. Biol. 57, 8271-8283 (2012).
25. G. Borasi, et al., Fast and high temperature hyperthermia coupled with radiotherapy as a possible new treatment for glioblastoma. J Ther Ultrasound 4 (2016).
26. Kibis, Buyuktahtakin, Optimizing multi-modal cancer treatment under 3D spatio-temporal tumor growth—ScienceDirect (2018) (May 3, 2021).
27. L. Hathout, B. Ellingson, W. Pope, Modeling the efficacy of the extent of surgical resection in the setting of radiation therapy for glioblastoma. Cancer Sci 107, 1110-1116 (2016).
28. V. Patel, L. Hathout, Image-driven modeling of the proliferation and necrosis of glioblastoma multiforme. Theor Biol Med Model 14 (2017).
29. S. Narasimhan, et al., Biophysical model-based parameters to classify tumor recurrence from radiation-induced necrosis for brain metastases. Med Phys 46, 2487-2496 (2019).
30. H. J. Bowers, E. E. Fannin, A. Thomas, J. A. Weis, Characterization of multicellular breast tumor spheroids using image data-driven biophysical mathematical modeling. Scientific Reports 10, 11583 (2020).
31. J. Ayensa-Jimenez, et al., Mathematical formulation and parametric analysis of in vitro cell models in microfluidic devices: application to different stages of glioblastoma evolution. Scientific Reports 10, 21193 (2020).
32. T. J. Sego, J. A. Glazier, A. Tovar, Unification of aggregate growth models by emergence from cellular and intracellular mechanisms. Royal Society Open Science 7, 192148 (2020).
33. K. F. Riley, M. P. Hobson, S. J. Bence, Mathematical Methods for Physics and Engineering: A Comprehensive Guide. Higher Education from Cambridge University Press (2006) https:/doi.org/10.1017/CB09780511810763 (Mar. 7, 2022).
34. S. Pasetto, R. A. Gatenby, H. Enderling, Bayesian Framework to Augment Tumor Board Decision Making. JCO Clinical Cancer Informatics, 508-517 (2021).
35. R. Ridgway, et al., Image segmentation with tensor-based classification of n-point correlation functions in MICCAI Workshop on Medical Image Analysis with Applications in Biology, (Citeseer, 2006).
36. K. Mosaliganti, et al., Tensor classification of N-point correlation function features for histology tissue segmentation. Med Image Anal 13, 156-166 (2009).
37. L. Cooper, J. Saltz, R. Machiraju, K. Huang, Two-Point Correlation as a Feature for Histology Images: Feature Space Structure and Correlation Updating. Conf Comput Vis Pattern Recognit Workshops, 79-86 (2010).
38. L. A. D. Cooper, J. H. Saltz, U. Catalyurek, K. Huang, Acceleration of Two Point Correlation Function Calculation for Pathology Image Segmentation in 2011 IEEE First International Conference on Healthcare Informatics, Imaging and Systems Biology, (2011), pp. 174-181.
39. B. J. Binder, M. J. Simpson, Spectral analysis of pair-correlation bandwidth: application to cell biology images. Royal Society Open Science 2, 140494 (2015).
40. S. M. Alessio, Digital Signal Processing and Spectral Analysis for Scientists: Concepts and Applications (Springer International Publishing, 2016) https:/doi.org/10.1007/978-3-319-25468-5 (Aug. 18, 2021).
41. P. J. E. Peebles, Statistical Analysis of Catalogs of Extragalactic Objects. I. Theory. The Astrophysical Journal 185, 413-440 (1973).
42. A. Nuttall, Some windows with very good sidelobe behavior. IEEE Transactions on Acoustics, Speech, and Signal Processing 29, 84-91 (1981).
43. S. Pasetto, H. Enderling, R. A. Gatenby, R. Brady-Nicholls, Intermittent Hormone Therapy Models Analysis and Bayesian Model Comparison for Prostate Cancer. Bull Math Biol 84, 2 (2021).

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A computer-implemented method for determining patient-specific tumor dynamics, the method comprising:
receiving an image of a tissue sample comprising a distribution of tumor cells and non-tumor cells;
applying a spatial correlation function to the image;
obtaining a power spectral density of the spatial correlation function of the image;
fitting the power spectral density of the spatial correlation function of the image to a power spectrum of a reaction-diffusion model; and
inferring a patient-specific tumor-dynamic coefficient based on the reaction-diffusion model of cancer.

2. The method of claim 1, wherein the patient-specific tumor-dynamic coefficient is a growth rate or a diffusion rate.

3. The method of claim 1, wherein the spatial correlation function is a 2-point correlation function or a 2-point cross-correlation function.

4. The method of claim 1, wherein the step of obtaining the power spectral density of the spatial correlation function of the image comprises applying a Fourier transform to the spatial correlation function of the image.

5. The method of claim 1, wherein the image is an immunofluorescence stained slide image, an immunohistochemistry (IHC) stained slide image, or a hematoxylin & eosin (H&E) stained slide image.

6. The method of claim 1, further comprising simulating a tumor's response to treatment using the patient-specific tumor-dynamic coefficient.

7. The method of claim 1, wherein the cancer is breast cancer, head and neck cancer, or prostate cancer.

8. The method of claim 1, wherein the tissue sample is a pre-treatment sample or a post-treatment sample.

9. The method of claim 1, wherein the step of receiving the image of the tissue sample comprises receiving a single image, and wherein the patient-specific tumor-dynamic coefficient is derived from the reaction-diffusion model of cancer using the single image.

10. A method of treating cancer in a subject, comprising:
receiving an image of a tissue sample comprising a distribution of tumor cells and non-tumor cells;
applying a spatial correlation function to the image;
obtaining a power spectral density of the spatial correlation function of the image;
fitting the power spectral density of the spatial correlation function of the image to a power spectrum of a reaction-diffusion model;
inferring a patient-specific tumor-dynamic coefficient based on the reaction-diffusion model of cancer;
determining one or more patient-specific tumor dynamic coefficients for the subject based on the reaction-diffusion model of cancer; and
guiding a treatment regimen for the subject using the one or more patient-specific tumor-dynamic coefficients.

11. The method of claim 10, wherein guiding the treatment regimen for the subject comprises increasing or decreasing an amount or frequency of treatment administered to the subject.

12. The method of claim 10, wherein the treatment regimen is one or more of chemotherapy, immunotherapy, or radiotherapy.

13. The method of claim 12, wherein the step of guiding the treatment regimen for the subject comprises selecting one or more of chemotherapy, immunotherapy, or radiotherapy based on the one or more patient-specific tumor-dynamic coefficients.

14. The method of claim 10, wherein the treatment regimen is a combination therapy.

15. The method of claim 10, further comprising administering the treatment regimen to the subject.

16. A system for determining patient-specific tumor dynamics, the system comprising:
at least one processor; and
at least one memory operably coupled to the at least one processor, wherein the at least one memory has computer-executable instructions stored thereon that, when executed by the at least one processor, cause the at least one processor to:
receive an image of a tissue sample comprising a distribution of tumor cells and non-tumor cells;
apply a spatial correlation function to the image;
obtain a power spectral density of the spatial correlation function of the image;
fit the power spectral density of the spatial correlation function of the image to a power spectrum of a reaction-diffusion model; and
infer a patient-specific tumor-dynamic coefficient based on the reaction-diffusion model of cancer.

17. The system of claim 16, wherein the patient-specific tumor-dynamic coefficient is a growth rate or a diffusion rate.

18. The system of claim 16, wherein the spatial correlation function is a 2-point correlation function or a 2-point cross-correlation function.

19. The system of claim 16, wherein the step of obtaining the power spectral density of the spatial correlation function of the image comprises applying a Fourier transform to the spatial correlation function of the image.

20. The system of claim 16, wherein the image is an immunofluorescence stained slide image, an immunohistochemistry (IHC) stained slide image, or a hematoxylin & eosin (H&E) stained slide image.

21. The system of claim 16, wherein the at least one memory has further computer-executable instructions stored thereon that, when executed by the at least one processor, cause the at least one processor to simulate a tumor's response to treatment using the patient-specific tumor-dynamic coefficient.

22. The system of claim 16, wherein the cancer is breast cancer, head and neck cancer, or prostate cancer.

23. The system of claim 16, wherein the tissue sample is a pre-treatment sample or a post-treatment sample.

24. The system of claim 16, wherein the step of receiving the image of the tissue sample comprises receiving a single image, and wherein the patient-specific tumor-dynamic coefficient is derived from the reaction-diffusion model of cancer using the single image.

25. (canceled)

26. (canceled)

27. (canceled)

28. (canceled)
